(19) United States
(12) Plant Patent Application Publication
(10) Pub. No.: US 2019/0141871 P1
(43) Pub. Date: May 9, 2019

(54) Apple tree named 'Bay 4029'

(50) ;

(71) Applicant: Michael Neumüller, Hallbergmoos (DE)

(72) Inventor: Michael Neumüller, Hallbergmoos (DE)

(21) Appl. No.: 16/350,365

(22) Filed: Nov. 8, 2018

Related U.S. Application Data
(60) Provisional application No. 62/707,577, filed on Nov. 8, 2017.

(51) Int. Cl. A01H 6/74

(52) U.S. Cl. PLT/161

(57) ABSTRACT

A new and distinct Malus domestica variety that bears fruits having a red skin color and good eating and keeping qualities.

### CROSS REFERENCE TO RELATED APPLICATION

This application claims the benefit of U.S. Provisional Patent Application Ser. No. 62/707,577, filed on Nov. 8, 2017, and entitled “Variety of Apple Tree Named ‘Bay 4029’”, the entire disclosure of which is incorporated herein by reference.

Botanical classification: Malus domestica.

Varietal denomination: ‘Bay 4029’.

### BACKGROUND OF THE INVENTION

The present invention comprises a new and distinct variety of apple tree, botanically known as Malus domestica, and referred to by the varietal name ‘Bay 4029’.

The new variety is the result of a planned breeding program conducted by the inventor in Hallbergmoos, Germany. The new variety is the result of a cross between ‘Rubinette’ (female parent, unpatented) and ‘Pomona’ (male parent, unpatented). ‘Bay 4029’ was first asexually reproduced in Hallbergmoos, Germany. The new variety is similar to ‘Rubinette’ in having highly aromatic fruits with a red skin color, but the fruits of ‘Bay 4029’ have firmer flesh and a longer storage time when compared to the fruits of ‘Rubinette’. The long storage time of the fruits of ‘Bay 4029’ is similar to ‘Pomona’, but the fruits of ‘Bay 4029’ have a red skin color versus the yellow-green and red skin color of the fruits of ‘Pomona’.

The following characteristics also distinguish the new variety from other varieties known to the inventor:
Striking red fruit color;
Firm and juicy fruits;
Long storage life;
Highly aromatic fruits with a hint of mango;
Late harvest time;
Little to no tendency for alternation; and
Simple and uncomplicated tree.

The new variety has been trial and field tested and has been found to retain its distinctive characteristics and remain true to type through successive asexual propagations.

### DESCRIPTION OF THE DRAWINGS

The accompanying photographic drawings illustrate the new cultivar, with the color being as nearly true as possible with color illustrations of this type. It should be noted that colors may vary with growing conditions and time of year:

FIG. 1 illustrates a close-up view of the mature fruit of the new variety; and

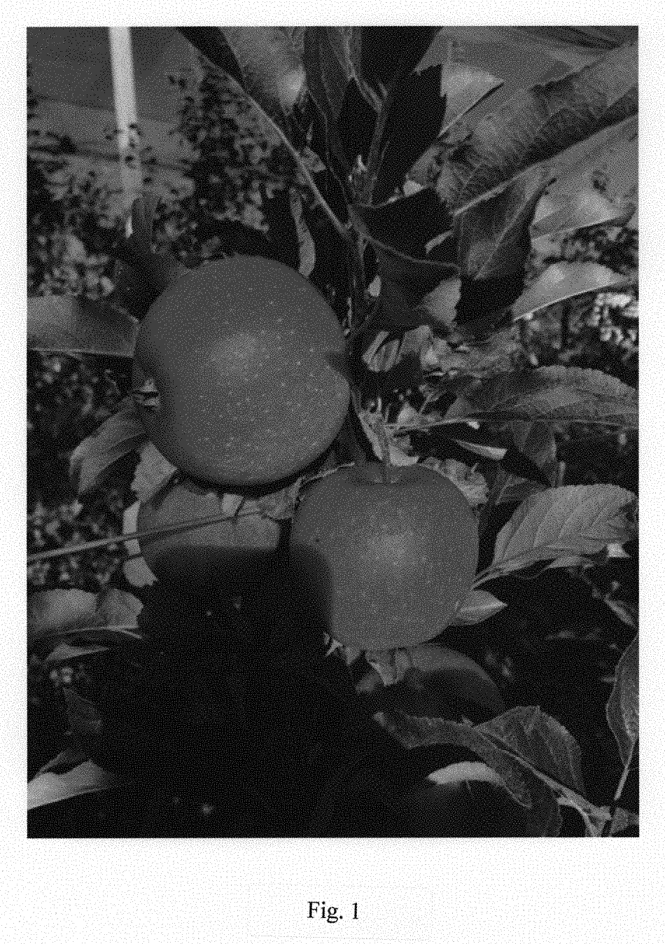

FIG. 2 illustrates multiple trees of the new variety.

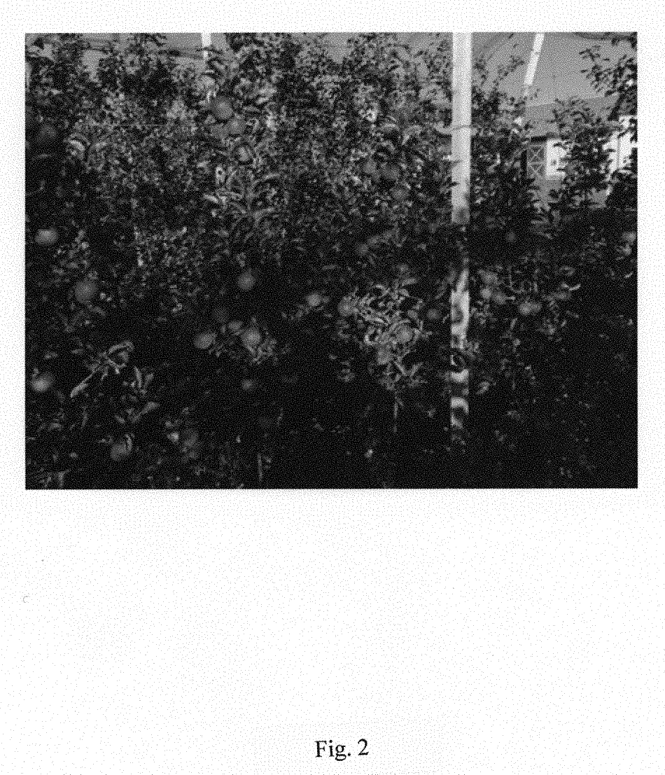

### DESCRIPTION OF THE PLANT

The following detailed description sets forth characteristics of the new cultivar. The new variety was grown under natural field conditions in Hallbergmoos, Germany.
Tree:
   Vigor.—Moderate.
   Branching.—Freely.
   Production.—Highly productive and regular with little to no alternance.
   Growth type.—Upright and slightly spreading.
   Bearing.—Annual, with thinning required.
   Flowers.—Bloom timing: Mid-late. Fertilization: Diploid pollination.
   Fruit.—Timing to maturity: Late — similar to ‘Braeburn’ (unpatented) or shortly thereafter. Approximately October in Hallbergmoos, Germany. Size: Medium. Shape: Elongated. Skin color: Red, with bright lenticels. Percentage of fruit overcolor: 70%. Flesh color: Cream. Flesh color: Cream. Flesh firmness: High (9.5 kg/cm²). Acidity: Low (3.5-5.3 g/l). Soluble dry matter: High (14-17% Brix). Flavor: Highly aromatic with a hint of mango.
   Keeping quality.—Extremely high. At temperatures of 1.5-2° C., apples are durable until June. In warehouse conditions, apples can be stored until September of the following year from picking.
   Disease resistance.—Moderate vulnerability to scab (similar to ‘Elstar’, U.S. Plant Pat. No. 6,450).

I claim:

1. A new and distinct variety of Malus domestica apple tree plant substantially as is herein described and illustrated.
